(19) United States
(12) Patent Application Publication
(10) Pub. No.: US 2024/0148942 A1
(43) Pub. Date: May 9, 2024

(54) SCAFFOLD FOR BONE REGENERATION AND MANUFACTURING METHOD THEREOF

(71) Applicants: PHARMAEXCEED S.R.L., Pavia (IT); INDUSTRIE BIOMEDICHE INSUBRI SA, MEZZOVICO-VIRA (CH)

(72) Inventors: Elia Bari, Pavia (IT); Giuseppe Perale, Pavia (IT); Sara Perteghella, Pavia (IT); Marzio Sorlini, Pavia (IT); Maria Luisa Torre, Pavia (IT)

(21) Appl. No.: 18/280,757

(22) PCT Filed: Mar. 9, 2022
(86) PCT No.: PCT/IB2022/052092

§ 371 Date: Sep. 7, 2023

(30) Foreign Application Priority Data
Mar. 9, 2021 (IT) 102021000005438

(51) Int. Cl. A61L 27/54; A61L 27/36; A61L 27/40; B33Y 10/00; B33Y 70/10; B33Y 80/00

(52) U.S. Cl. CPC A61L 27/54; A61L 27/3608; A61L 27/365; A61L 27/40; B33Y 10/00; B33Y 70/10; B33Y 80/00; A61L 2300/30; A61L 2430/02

(57) ABSTRACT

A scaffold for bone regeneration comprises a three-dimensional support structure made with a composite hybrid formed by a bovine bone-derived matrix reinforced with a polymer and containing collagen fragments; the support structure is functionalized with secretome, in particular mesenchymal stem cell secretome, in lyophilized form (lyosecretome).

### CROSS-REFERENCE TO RELATED APPLICATIONS

This patent application claims priority from Italian Patent Application No. 102021000005438 filed on Mar. 9, 2021, the entire disclosure of which is incorporated herein by reference.

### TECHNICAL FIELD

The present invention relates to a scaffold for bone regeneration.

In particular, the present invention falls within the field of tissue engineering as multidisciplinary sector that applies the principles of engineering and of biology to produce biological replacements capable of restoring tissues, for example, bone tissue, invalidated by disease, trauma or as a result of aging. More precisely, the technical field of the present invention refers to a scaffold (i.e., a typically three-dimensional scaffold designed for cell support) loaded (even only partly) with cell-derived functional material, whose release promotes the regeneration of bone tissue, invalidated by disease, trauma or as a result of aging.

### STATE OF THE PRIOR ART

Bone tissue, the main constituent of the skeleton, is a highly specialized mineralized connective tissue, with important structural and metabolic functions. Through a process called “bone remodeling”, bone has the inherent ability to regenerate as part of the repair process in response to an injury, and also during skeletal development or continuous remodeling throughout adult life. At times, the physiological process of bone regeneration is impaired or insufficient, for example due to infections, osteonecrosis, trauma, tumors or inherent genetic diseases. In these cases, bone grafting is a widely used therapeutic strategy.

Although widely practised, both autologous bone grafting (which involves the transplant of fresh cortical or trabecular bone, or a combination of both, from one site in the body to another of the patient's body), and allogenic bone graft (transplant from donor) have limits and problems.

Consequently, bone substitutes of biological and synthetic origin have been developed. Examples of synthetic materials are bioactive glasses, resorbable hydroxyapatite (HA), β-tricalcium phosphate (TCP) and combinations thereof. They have been proposed for the similarity to the bone mineral matrix, but, at the current state of the art, synthetic bone substitutes that have at least the same biological or mechanical properties with respect to bone do not exist. On the other hand, materials of animal origin (xenografts) can provide structures similar to living tissues and therefore induce specific cellular responses.

Materials that combine natural and synthetic components, such as a biohybrid bone substitute composed of a bovine bone-derived matrix reinforced with poly(L-lactide-co-ε-caprolactone) (PLCL), have also be proposed. Recently, scaffolds constructed with this material were proposed as bone substitutes for oral and maxillofacial surgeries and dental implantology.

The aforesaid materials are used, in particular, to construct scaffolds for bone regeneration.

With the term scaffold we mean as a support, commonly three-dimensional and often porous, made with biocompatible and optionally bioresorbable and/or biodegradable material, having the capacity to temporarily substitute natural tissue promoting mechanisms of differentiation, adhesion and proliferation of the cells loaded thereon. During production of the extracellular matrix by the cells, the scaffold tends to biodegrade and, simultaneously, to be substituted by the regenerated biological tissue, thereby restoring the impaired function and, at the same time, avoiding surgical reintervention for removal of the prosthetic element.

Loading a scaffold with active substances, mainly of cellular origin, is also known. The cells used can be of various types, for example: autologous, allogenic, xenogenic and stem cells (both embryonic and adult). Among adult stem cells, mesenchymal stem cells (MSCs) currently represent a valuable resource in tissue engineering. In fact, MSCs can be easily isolated from the patient, in particular from bone marrow or from adipose tissue, and expanded in culture to a physiologically relevant number. Moreover, MSCs have the ability to differentiate into different cell lines, such as, for example, osteoblasts, chondrocytes, myocytes and adipocytes, giving rise to various cell tissues, such as, for example, bone, cartilage, tendon, muscles and adipose tissue.

Current bone tissue engineering, based on three-dimensional scaffolds loaded with stem cells, including MSCs, has some limits, for example connected to the administration of cells, such as rejection reactions, induction of tumors and transmission of infections. Furthermore, the handling of the stem cells involves strict control programs and elaborate protocols to standardize the production and storage of large numbers of stem cells.

Furthermore, the production of scaffolds loaded with stem cells is relatively complex and problems can be encountered in obtaining a suitable distribution and adhesion of the stem cells on the scaffold.

### SUBJECT MATTER OF THE INVENTION

An object of the present invention is to provide a scaffold for bone tissue regeneration, and the manufacturing method thereof, that overcomes the drawbacks described in the known art.

In particular, an object of the invention is to provide a scaffold that allows an improved and controlled reconstruction of damaged bone tissue. A further object of the invention is to provide a particularly efficient, simple and inexpensive method for manufacturing said scaffold.

In accordance with these objects, the present invention therefore relates to a scaffold for bone tissue regeneration and manufacturing method thereof as defined in basic terms in the appended claims 1 and 8, respectively.

Further preferred characteristics of the invention are indicated in the dependent claims.

In substance, the scaffold according to the invention consists of a composite structure, formed by a bovine bone matrix and by a biodegradable polymer, for example PLCL, integrating mesenchymal stem cell secretome (MSC-secretome), in particular lyophilized secretome (lyosecretome).

In fact, loading the scaffolds with secretome makes it possible to produce a bone substitute with improved osteoinductive ability and without the problems linked to the use of stem cells, as is instead the case with traditional engineered tissues. Moreover, unlike stem cells, secretome, in particular in the form of lyosecretome, can be prepared in advance to be ready for use in the case of acute diseases that require prompt treatment.

Experimental results confirm that the scaffold according to the invention ensures bone tissue regrowth with improved speed and quality of the new bone formation compared to the known art.

Other characteristics of the invention, implemented in the preferred embodiments, are also particularly advantageous compared to conventional engineered tissues that combine stem cells and scaffolds.

Preferably, the MSC-secretome used is lyosecretome obtained by ultrafiltration and lyophilization, with mannitol (or another equivalent substance) as cryoprotectant.

In accordance with a preferred embodiment of the invention, lyosecretome is loaded onto the scaffold made of bovine bone tissue by immersion and subsequent lyophilization, resulting in the formation of a biologically active coating on the surfaces of the material.

The adsorption method is simple, direct and inexpensive to load the active substance (lyosecretome) onto the scaffold.

To ensure efficient loading and also efficient release of the active substance, the lyosecretome formulation is prepared with specific excipients.

In particular, Lutrol® F127 (poloxamer 407) and NaCl are added to the lyosecretome solution to be used for adsorption on the scaffold.

Poloxamer 407 is used to increase the wettability of the support structure and promote penetration of the lyosecretome solution into the internal pores of the support structure to obtain homogeneous lyosecretome loading. In addition to promoting homogeneous lyosecretome loading, poloxamer 407, like mannitol, stabilizes the proteins contained in the secretome, preventing its denaturation. On the other hand, the introduction of NaCl prevents crystallization of the mannitol on the surface of the support structure during the lyophilization process.

### BRIEF DESCRIPTION OF THE DRAWINGS

Further characteristics and advantages of the present invention will be evident from the description of the following non-limiting embodiments, with reference to the figures of the accompanying drawings, wherein:

FIG. 1 shows SEM (Scanning Electron Microscopy) images of reference scaffolds (non-functionalized: images a, b, c) and of scaffolds in accordance with the invention (functionalized with lyosecretome: images d, e, f, g, h, i) with increasing enlargements (30× and 100×);

FIG. 2 is a graph representing the release kinetics of the functional substances (proteins and lipids) from scaffolds in accordance with the invention (in vitro test; scaffolds immersed in PBS at pH 7.2 and room temperature);

FIGS. 3A to 3D are enlarged images (enlargement 4×) that show the cell growth on a control scaffold, not functionalized with lyosecretome, and a scaffold in accordance with the invention functionalized with lyosecretome: A, B show, respectively, the control scaffold (CTRL SB) and the scaffold of the invention (CTRL SBlyos) without cell growth; C, D show the growth of SVF at 14 days respectively on the control scaffold (SB+SVF OM) and on the scaffold of the invention (SB+SVF OM);

FIGS. 4A to 4D are enlarged images (enlargement 20×) that show the cell growth, highlighted by H&E staining, after 60 days on a control scaffold, not functionalized with lyosecretome (SB+SVF OM), and on the scaffold in accordance with the invention functionalized with lyosecretome (SBlyos+SVF OM).

### PREFERRED EMBODIMENT OF THE INVENTION

The scaffold according to the invention comprises a three-dimensional support structure formed by a bovine bone-derived matrix, by a synthetic polymer, in particular poly(L-lactide-co-ε-caprolactone) (PLCL), and by collagen fragments, in particular collagen type I of animal origin.

In particular, the scaffold is a composite scaffold having a bovine bone matrix, shaped (i.e., formed by a piece of bovine bone shaped in a predefined shape) or ground (i.e., formed by compacted and consolidated ground bovine bone) and coated with polymer material and containing collagen fragments.

Scaffolds of this type, usable in accordance with the invention, are currently marketed as Class III medical devices according to the Directive 93/42/EEC, in particular with the trade name SmartBone®, and have recently been proposed as bone substitutes for oral and maxillofacial surgery, for dental implants, for orthopedic and oncological applications. Details on these scaffolds are available in the literature, for example in:
WO2010070416A1;
Grecchia F. et al. “Reconstruction of the Zygomatic Bone with SmartBone®: Case Report”—Journal of Biological Regulators and Homeostatic Agents 2015, 29, 42-47;
Mandelli F. et al. “Clinical and Histological Evaluation of Socket Preservation Using SmartBone®, a Novel Heterologous Bone Substitute: a Case Series Study”—Oral Implantology 2018, XI);
Ferracini R. et al. “Composite Xenohybrid Bovine Bone-Derived Scaffold as Bone Substitute for the Treatment of Tibial Plateau Fractures”—Appl. Sci. 2019, 9, 2675; doi:10.3390/app9132675;
Boffano M. et al. “A Preliminary Study on the Mechanical Reliability and Regeneration Capability of Artificial Bone Grafts in Oncologic Cases, With and Without Osteosynthesis”-J. Clin. Med. 2020, 9, 1388; doi:10.3390/jcm9051388.

Advantageously, the scaffold is treated so as to integrate collagen fragments containing RGD (arginylglycylaspartic acid) for the purpose of promoting cell vitality, hydrophobicity of the matrix and biocompatibility in general, as illustrated in Pertici G. et al. “Composite Polymer-coated Mineral Scaffolds for Bone Regeneration: from Material Characterization to Human Studies”—J. Biol. Regul. Homeost. Agents, 2015; 29(3 Suppl 1):136-48; and in Cingolani A. et al. “Improving Bovine Bone Mechanical Characteristics for the Development of Xenohybrid Bone Grafts”—Current Pharmaceutical Biotechnology 2018, 19, 1005-1013, doi:10.2174/1389201020666181129115839.

In accordance with the invention, the support structure is loaded and hence functionalized with lyophilized secretome (lyosecretome), in particular mesenchymal stem cell secretome, defining a functional material carried by the support structure.

The support structure can have various shapes, sizes and geometries, also according to the specific intended use of the scaffold.

The scaffold can be made according to one of the available known techniques, such as a 3d printing process, or it can be shaped from a piece of bovine bone treated with the polymer and the collagen fragments, or by consolidating and compacting the ground bovine bone together with the polymer and the collagen fragments in a mold.

Subsequently, the scaffold is functionalized with secretome by adsorption, through immersion of the scaffold in a solution containing the secretome.

The secretome present on the support structure is in particular mesenchymal stem cell secretome (MSC-secretome).

Advantageously, the MSC-secretome is isolated and purified from supernatants of mesenchymal stem cells (MSC) in a culture using a combined process of ultrafiltration and lyophilization described in the patent application WO2018/078524.

In brief, the process for obtaining the secretome includes the steps of:
(i) collecting from mesenchymal stem cells in culture a supernatant, comprising both a soluble fraction (essentially proteins) and an insoluble fraction in particle form (vesicles),
(ii) dialyzing or ultrafiltering the biologic fluid using a membrane having a threshold value (cut-off) equal to or smaller than 500,000 Dalton and a dialysis or ultrafiltration fluid,
(iii) adding a cryoprotectant to the dialyzed or ultrafiltered solutions obtained, and
(iv) lyophilizing the resulting solution.

To prevent undesirable effects, such as breaking up of the extracellular vesicles and instability of the proteins, caused by rapid freezing and by drying during the lyophilization process, a cryoprotectant, preferably mannitol, is added to the ultrafiltered supernatant. At the end of the process a lyophilized powder, namely lyosecretome (lyophilized secretome), which contains the secretome obtained from mesenchymal stem cells (MSCs), is obtained.

In a preferred embodiment, lyosecretome is loaded onto the support structure by immersion of the support structure in a solution containing lyosecretome, followed by subsequent lyophilization, so that the surfaces of the support structure are coated with lyosecretome. Specifically, two excipients, such as Lutrol® F127 (poloxamer 407) and NaCl, are added to the lyosecretome solution. Poloxamer 407 is used to increase the wettability of the support structure and promote penetration of the lyosecretome solution into the internal pores of the support structure to obtain homogeneous loading of lyosecretome. Besides promoting homogeneous loading of lyosecretome, poloxamer 407, just as mannitol, stabilizes the proteins contained in the secretome, preventing denaturation.

On the other hand, the addition of NaCl prevents mannitol crystallization on the surface of the support structure during the lyophilization process.

### EXAMPLES

Some experimental examples that describe the manufacture and efficiency of scaffolds in accordance with the invention are set forth below.

1. Lyosecretome Preparation

Lyosecretome (lyophilized secretome of mesenchymal stem cells) was prepared according to the methods indicated above, substantially as set forth in WO2018/078524.

In particular, lyosecretome obtained from adipose-derived cells (AD-MSC) collected from adipose tissues, was used. The cells were cultivated using standard techniques. Secretome release from the cells was obtained by cultivating the cells DMEM/F12 without platelet lysate for 48 h; the conditioned media were collected after 9, 24, 33 and 48 hours and grouped.

The MSCs were detached with trypsin-EDTA and tested to evaluate cell vitality and compliance with all the requirements necessary for clinical use.

The conditioned media were centrifuged at 3500 rpm for 10 min to eliminate cell debris and apoptotic bodies, obtaining supernatants that were collected and ultrafiltered by tangential flow filtration. Both the free soluble proteins and the vesicles produced by the MSCs were maintained.

The samples were initially concentrated at 0.5×10⁶ cellular equivalents per mL (calculated by dividing the total number of cells and the mL of concentrated and purified supernatant) and then diafiltered using sterilized ultrapure water.

Mannitol (final concentration 0.5% w/v) was dissolved in the concentrated and purified secretome; the resulting solution was frozen at −80° C. and lyophilized at 8×10⁻¹ mbar and −50° C. for 72 h.

The lyosecretome obtained was stored at −20° C. until use. Each mg of lyosecretome corresponds to 0.1×10⁶ cellular equivalents (calculated by dividing the total number of cells used for production and the milligrams of lyosecretome obtained).

A protein content of 30.72±2.139 μg and a lipid content of 1.62±0.0329 μg per mg of lyophilized powder (mean value±standard deviation obtained from three independent replications starting from the same batch of lyosecretome) was determined in the lyosecretome obtained.

2. Preparation of Scaffolds Functionalized with Lyosecretome

Scaffolds were made according to the method previously reported in the literature (in particular in WO2010070416A1 and in Pertici G. et al. “Composite polymer-coated mineral scaffolds for bone regeneration: from material characterization to human studies”—J. Biol. Regul. Homeost. Agents, 2015; 29(3 Suppl 1):136-48).

Bovine-derived spongy bone certified for human use and free from bovine spongiform encephalopathy was used. The scaffolds were then obtained by reinforcing the bovine bone-derived matrix with a mixture of PLCL and collagen fragments containing RGD. After final packaging in two layers, the product was sterilized with ethylene oxide.

Subsequently, the scaffolds were functionalized with lyosecretome, in particular by adsorption.

Each sample scaffold (dimensions 1 cm×1 cm×0.3 cm) was loaded with 16,000 cellular equivalents of lyosecretome.

The samples were individually immersed in an aqueous solution containing lyosecretome (1.7 mg/mL), Lutrol® F127 (0.1% w/v) and NaCl (0.1% w/v) for 1 hour at 4° C.

Lutrol® F127 was added as surfactant to increase the wettability of the support structure and thus enable more homogeneous lyosecretome loading, allowing the solution containing lyosecretome to penetrate into the support structure. NaCl was added to reduce mannitol crystallization during the lyophilization process.

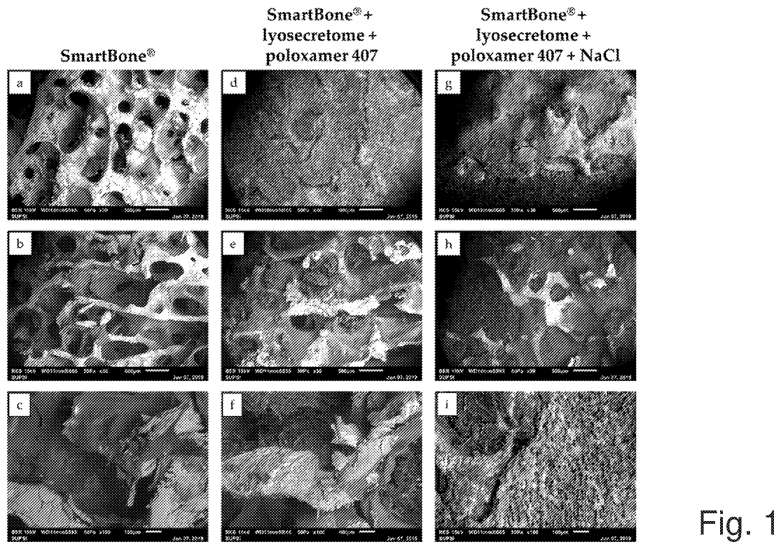

FIG. 1 shows the SEM morphological study of:
scaffolds in accordance with the invention loaded with lyosecretome with the addition of poloxamer 407 (images d, e, f);
scaffolds in accordance with the invention loaded with lyosecretome with the addition of poloxamer 407 and NaCl (images g, h, i);
control scaffolds, not loaded with lyosecretome (images a, b, c).

The SEM images were taken with increasing enlargements (the first two images of each group with enlargement 30×, the third with enlargement 100×).

Morphological characterization showed the deposition of a lyosecretome coating on the surfaces of the material of the support structure (images d, e, f). The external pores were almost completely closed, as were the internal pores (with a few exceptions in the center, image e).

The addition of NaCl as excipient to prevent mannitol crystallization proved advantageous also from the viewpoint of lyosecretome deposition. In fact, using a formulation with poloxamer 407 and NaCl a homogeneous deposition of lyosecretome on the surface of the support structure (image g), with complete closing of the external pores and also of the central pores (images h, i), was observed.

The samples were then frozen at −80° C. and lyophilized at 8×10¹ mbar and −50° C. for 24 h.

For determination of the protein/lipid load, the samples were dispersed in deionized water under magnetic stirring for 96 h.

The solutions obtained were tested to determine the protein and lipid content. The average protein and lipid content was equal to 657.25±6.662 μg and 18.44±2.452 μg, respectively.

3. Release of Active Substance

The scaffold samples were tested by immersion in phosphated buffered saline (PBS) at pH 7.2 to evaluate the speed of release of the functional substances, in particular proteins and lipids, incorporated in the lyosecretome.

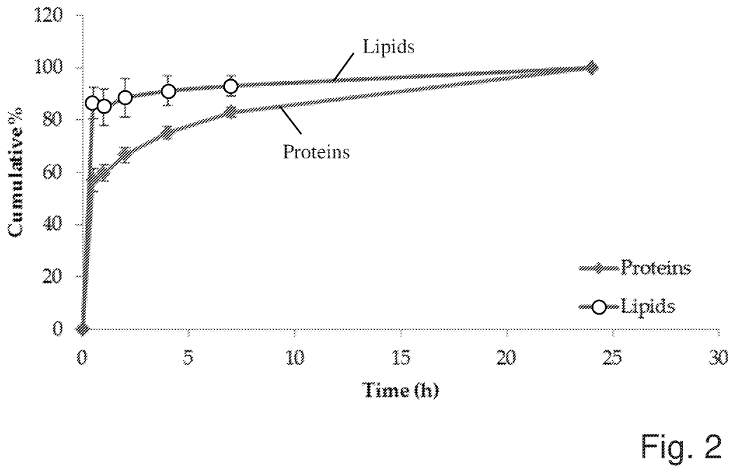

The protein and lipid release profiles are shown in FIG. 2. The cumulative release from the scaffolds showed a concentrated initial release, in 30 minutes, of 57% for the proteins and of 86% for the lipids. 100% of the proteins and of the lipids was released after 24 hours. This rapid release is presumably due to the components of the formulation (mannitol, poloxamer 407 and NaCl) which being hydrophilic compounds, easily recall the PBS into the scaffold and dissolve.

Complete release of the secretome from the scaffold after the times indicated above was confirmed by SEM study.

4. Stromal Vascular Fraction (SVF) Tissue Growth

For comparison purposes, stromal vascular fraction tissue growth was evaluated on scaffolds in accordance with the invention, functionalized with lyosecretome, and reference scaffolds, identical but not functionalized with lyosecretome. Growth was evaluated at 14 days and at 60 days.

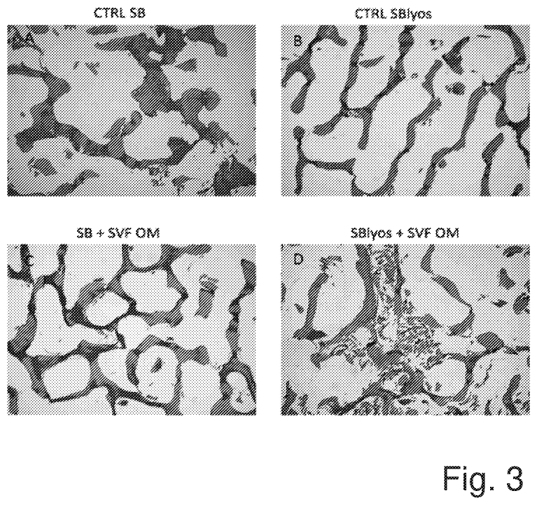

FIGS. 3A and 3B are the initial images, before cell growth, respectively of a non-functionalized scaffold and a scaffold functionalized with lyosecretome. H&E staining was performed, which shows the trabecular structure of the scaffolds without cells (FIGS. 3A and 3B, respectively) and colonization of the scaffold by SVF, showing an initial formation of new tissue at 14 days (FIG. 3C and FIG. 3D), mainly on the sample functionalized with lyosecretome.

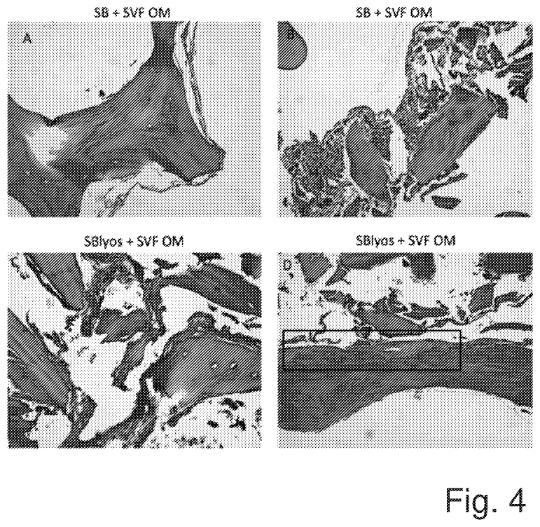

After 60 days, the presence of new bone tissue was evident both on control scaffolds and on the scaffold of the invention (functionalized with lyosecretome) (FIGS. 4A-D). In all the samples, SVF had colonized and formed new tissue, starting from the periphery (FIG. 4A) of the scaffold and filling the lacunae in the bones (FIG. 4B). Even if the area of formation of the new tissue could be detected on both the scaffolds, the growth was more marked on the functionalized sample, as shown in FIGS. 4C and 4D, respectively.

The increased formation of well organized bone tissue can thus be attributed to the presence of lyosecretome.

To further confirm this result, staining was performed with collagen 1 (COLL-1), osteocalcin (OCN) and transforming growth factor beta (TGFβ) both on the non-functionalized scaffolds and on the scaffolds functionalized with lyosecretome. The expression of these proteins by cells grown on the scaffolds was controlled at 60 days. OCN and TGFβ were highly expressed, while COLL-1 was weakly positive. Nonetheless, COLL-1 markedly stained the new tissue on the functionalized scaffold.

All the results confirm that although the formation of bone tissue is observed in all the scaffolds tested (functionalized and non-functionalized), the scaffolds in accordance with the invention show greater and faster growth of bone tissue.

Ultimately, the experimental tests confirm that a scaffold made of a bovine matrix and of a biodegradable polymer (PLCL) and containing collagen fragments and functionalized with lyosecretome promotes a faster and more effective growth of bone tissue compared to the same non-functionalized scaffold.

After having deposited SVF on the functionalized and non-functionalized scaffolds, the growth of the tissue was monitored at 14 and 60 days, showing increasing areas of new tissue over time. After 14 days, a greater cell proliferation was observed on the scaffold functionalized with lyosecretome, where an initial filling of the lacunae between the trabeculae with cells was detected. On the other hand, on the control scaffolds (without lyosecretome), the process was slower and the tissue formation was less organized at 60 days. On both the scaffolds, the cells were differentiated into osteoblasts and were able to mineralize after 60 days. However, the scaffolds of the invention (with lyosecretome) showed a higher expression of osteoblast markers and a higher quantity of newly formed trabeculae compared to the control scaffolds. Quantification analysis of the newly formed mineralized tissue showed that lyosecretome induces bone formation more effectively. Furthermore, immunohistochemical analysis performed at 60 days made it possible to observe the presence of highly organized bone tissue on the scaffolds with lyosecretome, indicative of cells stimulated to grow and differentiate.

1. A scaffold for bone regeneration, comprising a support structure made with a composite hybrid formed by a bovine bone-derived matrix reinforced with at least one polymer and containing collagen fragments; the support structure being functionalized with secretome.

2. The scaffold according to claim 1, wherein the secretome is mesenchymal stem cell secretome.

3. The scaffold according to claim 1, wherein the secretome is lyosecretome (lyophilized secretome).

4. The scaffold according to claim 1, wherein the secretome contains both a soluble fraction, in particular comprising proteins, and an insoluble particulate fraction, in particular extracellular vesicles.

5. The scaffold according to claim 1, wherein the secretome contains proteins and lipids.

6. The scaffold according to claim 1, wherein the bovine bone-derived matrix is reinforced with poly (L-lactide-co-s-caprolactone) (PLCL).

7. The scaffold according to claim 1, wherein the collagen fragments are of animal origin.

8. A method for manufacturing the scaffold for bone regeneration according to claim 1, comprising the steps of: preparing the support structure by 3D printing; and loading the support structure with the secretome.

9. The manufacturing method according to claim 8, wherein the step of loading the support structure with the secretome is carried out by adsorption of the secretome onto the support structure.

10. The manufacturing method according to claim 9, wherein the secretome is loaded onto the support structure by immersion of the support structure in a solution containing the secretome, and subsequent lyophilization.

11. The manufacturing method according to claim 9, wherein the secretome is in solution with poloxamer 407 and/or NaCl.
